(19) United States
(12) Patent Application Publication
(10) Pub. No.: US 2007/0298133 A1
(43) Pub. Date: Dec. 27, 2007

(54) Compositions and kits comprising a melatonin component and a flavanol component

(75) Inventors: Jesus Velazquez, West Chester, OH (US); Amy Violet Trejo, Oregonia, OH (US); James Patrick Ebel, Lebanon, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/820,370

(22) Filed: Jun. 19, 2007

Related U.S. Application Data
(60) Provisional application No. 60/816,075, filed on Jun. 23, 2006.

(51) Int. Cl. A61K 31/403; A61K 36/82; A61P 43/00

(52) U.S. Cl. 424/729; 514/415

(57) ABSTRACT

Disclosed herein are compositions and kits, wherein each composition comprises a melatonin component, a flavanol component, or both, and wherein each kit comprises a melatonin component and a flavanol component. The compositions and kits are useful for restorative sleep function and enhanced energy. Further disclosed herein are methods of using the compositions and kits.

### CROSS REFERENCE TO RELATED APPLICATIONS

This application claims the benefit of U.S. Provisional Application No. 60/816,075, filed on Jun. 23, 2006.

### FIELD OF THE INVENTION

The present invention is directed to compositions and kits, wherein each composition comprises a melatonin component, a flavanol component, or both, and wherein each kit comprises a melatonin component and a flavanol component. The compositions and kits are useful for restorative sleep function and enhanced energy.

### BACKGROUND OF THE INVENTION

One of the functions of sleep is the maintenance, restoration, and repair of the body. Sleep is generally characterized by anabolic activity (including building and remodeling) in muscle, bone, connective tissue, skin, and major organs including the brain. One result of this activity is restoration of function including physical and mental performance such as stamina, energy, and mental alertness.

Supplements, foods, medicines, and other products that are used to induce and improve sleep with respect to duration and quality are commercially available. Examples are supplements such as melatonin, valerian and hops, over-the-counter medications like diphenhydramine and doxylamine, and prescription medications such as AMBIEN® and LUNESTA®.

However, these products fail to provide any additional benefits beyond induction and improvement of sleep, and the indirect benefits derived from such induction and improvement. It would be beneficial to utilize the sleep cycle to more directly induce these benefits, when the body is susceptible to building and remodeling activities, such that the benefits are achieved and an even greater level than would otherwise be achieved.

### SUMMARY OF THE INVENTION

The present invention is directed to compositions and kits useful for restorative sleep function and enhanced energy.

In particular, the invention is directed to a composition comprising:
(a) a melatonin component; and
(b) a flavanol component.

The invention is further directed to a kit comprising:
(a) a composition comprising a melatonin component; and
(b) a composition comprising a flavanol component.

The invention is further directed to methods of using the compositions and kits.

### DETAILED DESCRIPTION OF THE INVENTION

The present invention is directed to combinations of a melatonin component and a flavanol component, whether such components are combined in a single composition or in discrete compositions within a kit. The compositions and kits, and methods of use thereof, are useful for more directly inducing the benefits of restorative sleep, particularly enhanced energy.

Various documents including, for example, publications and patents, are recited throughout this disclosure. All such documents are hereby incorporated by reference.

Trade names for products or components including various ingredients may be referenced herein. The inventors herein do not intend to be limited by materials under a certain trade name.

In the description of the invention various embodiments or individual features are disclosed. As will be apparent to the ordinarily skilled practitioner, all combinations of such embodiments and features are possible and can result in preferred executions of the present invention.

The compositions herein may comprise, consist essentially of, or consist of any of the elements as described herein.

While various embodiments and individual features of the present invention have been illustrated and described, various other changes and modifications can be made without departing from the spirit and scope of the invention. As will also be apparent, all combinations of the embodiments and features taught in the foregoing disclosure are possible and can result in preferred executions of the invention.

With respect to dosing preferences, dosage levels are developed based on typical human subjects (e.g., a 65 kg subject). Wherein the present composition is used in other mammals or in various human subjects, it may be necessary to modify the dosage. Modification of dosages based on the needs of the subject is well within the skill of the ordinary artisan. It is therefore understood that these dosage ranges are by way of example only, and that daily administration can be adjusted depending on various factors. The specific dosage of the compound to be administered, and the duration of treatment are interdependent. The dosage and treatment regimen will also depend upon such factors as the specific compound used, the treatment indication, the efficacy of the compound, the personal attributes of the subject (such as, for example, weight, age, gender, and medical condition of the subject), and compliance with the treatment regimen.

The present invention is directed to compositions and kits, wherein each composition comprises a melatonin component, a flavanol component, or both, and wherein each kit comprises a melatonin component and a flavanol component. The compositions and kits are useful for restorative sleep function and energy enhancement.

The melatonin component and the flavanol component are described as follows. These compounds may be utilized together in a composition, or may be provided in separate compositions as part of a kit. In particular, dosing convenience may be provided in embodiments wherein each of these compounds is present in one composition, while convenience and further dosing flexibility may be provided wherein each of these compounds is present in separate compositions as part of the kit. For example, the user of the kit may be in need of only one of the melatonin component and the flavanol component on any given day, and therefore excess dosing of unnecessary compounds during administration may be avoided.

The melatonin component and the flavanol component are described as follows. In addition, optional dosage guidance is provided, as well as optional adjuncts and dose forms.

### The Melatonin Component

The present kits and compositions comprise a melatonin component. As used herein, the melatonin component includes melatonin, melatonin precursors, melatonin agonists, and compounds that raise endogenous melatonin levels, as well as mixtures thereof. See e.g., U.S. Patent Publication 2004/0044064.

Melatonin components, including melatonin, are commercially available. Non-limiting examples of melatonin precursors, melatonin agonists, and such other compounds that mimic melatonin activity, include N-acetyl-5-hydroxytryptamine. For example, these may include compounds that compete with melatonin at the melatonin receptor and compounds that stimulate melatonin receptors to have an effective opposite to that of melatonin (melatonoin inverse agonists), in addition to drugs (melatonin blockers or melatonin stimulants) and interventions (such as exposure to light or darkness) that lower or raise, respectively, endogenous melatonin levels.

In one embodiment herein, the melatonin component is melatonin.

In one embodiment herein, a composition comprising a melatonin component comprises a dosage of about 0.01 mg to about 100 mg the melatonin component, alternatively from about 0.1 mg to about 10 mg of the melatonin component, and alternatively from about 0.1 mg to about 1 mg of the melatonin component. The ordinarily skilled artisan will adjust the dose to effect the desired change in phase of the circadian rhythm of endogenous melatonin production.

Since melatonin components, particularly melatonin, may be absorbed across almost all tissues, many routes of administration are possible. These include but are not limited to submucosal, sublingual, intranasal, ocular cul-de-sac, rectal, transdermal, buccal, intravenous, intramuscular, and subcutaneous routes of administration. A variety of administration means, including but not limited to capsules, tablets, suppositories, or any reservoir capable of containing and dispensing melatonin, are useful. In a preferred embodiment herein, the composition comprising the melatonin component is administered orally.

Typically, the composition comprising the melatonin component is administered in a manner commensurate with desired onset of sleep. In one embodiment, the composition comprising the melatonin component is administered within 1 hour of desired sleep time, at least about 1 hour of desired sleep time, at least about 4 hours prior to desired sleep time, or at least about 8 hours prior to desired sleep time. Compositions in sustained or delayed release form may typically be administered at least about 4 hours or at least about 8 hours prior to desired sleep time. In one embodiment, administration preferably follows the descriptions set forth in the following documents: U.S. Pat. Nos. 5,242,941; 5,420,152; 5,591,768; 5,707,652; 5,716,978; 6,069,164; 6,423,738; 6,638,963; and 6,794,407; and U.S. Patent Publication Nos. 2004/0044064 and 2003/0008912.

### The Flavanol Component

The flavanol component utilized herein may be useful for a variety of purposes, including a source of additional energy for those in need thereof. Flavanol components are quite well-known in the art, and the ordinarily skilled artisan has the capability to choose any such component for use in the present invention.

Flavanols are natural substances present in a variety of plants (e.g., fruits, vegetables, and flowers). The flavanols which may be utilized in the present invention can be extracted from, for example, fruit, vegetables, green tea or other natural sources by any suitable method well known to those skilled in the art. For example, extraction with ethyl acetate or chlorinated organic solvents is a common method to isolate flavanols from green tea. Flavanols may be extracted from either a single plant or mixtures of plants. Many fruits, vegetables, and flowers contain flavanols but to a lesser degree relative to green tea. Plants containing flavanols are known to those skilled in the art. Examples of the most common flavanols which are extracted from tea plants and other members of the Catechu gambir (Uncaria family) include, for example, catechin, epicatechin, gallocatechin, epigallocatechin, epicatechin gallate, and epigallocatechin gallate.

The flavanols utilized in all compositions of the present invention can be in the form of a tea extract. The tea extract can be obtained from the extraction of unfermented teas, fermented teas, partially fermented teas, and mixtures thereof. Preferably, the tea extracts are obtained from the extraction of unfermented and partially fermented teas. The most preferred tea extracts are obtained from green tea. Both hot and cold extracts can be used in the present invention. Suitable methods for obtaining tea extracts are well known. See e.g., Ekanayake, U.S. Pat. No. 5,879,733, issued Mar. 9, 1999; Tsai, U.S. Pat. No. 4,935,256, issued June, 1990; Lunder, U.S. Pat. No. 4,680,193, issued July, 1987; and Creswick, U.S. Pat. No. 4,668,525, issued May 26, 1987.

The preferred source of flavanols in the compositions of the present invention is green tea. Wherein green tea, and in particular the flavanols present in green tea, are incorporated into the composition or kit, the present inventors have discovered that the flavanols are at least partially responsible for delaying the bioavailability of bracers such as caffeine, which contributes to the reduction and/or elimination of nervousness and tension typically associated with such bracers.

Alternatively, these same flavanols may be prepared by synthetic or other appropriate chemical methods and incorporated into the present compositions. Flavanols, including catechin, epicatechin, and their derivatives are commercially available.

The amount of flavanols in the compositions of the present invention can vary.

### Kits of the Present Invention

In one embodiment herein, the kits comprise a composition comprising the melatonin component and a composition comprising the flavanol component. In this embodiment, the kits comprise at least two discrete compositions, i.e., at least two compositions that are compositionally distinct.

In this embodiment, the kits are particularly advantageous to different needs regarding time of dosing of the compositions. For example, it may be advantageous to administer the composition comprising the melatonin composition at a time relative to desired sleep time of the mammal, for example, within about 1 hour, at least about 1 hour, at least about 4 hours or at least about 8 hours prior to desired sleep time. Such time of administration may be dependent upon a variety of factors, such as whether the composition is formulated as an immediate, sustained, or delayed release formulation, or dependent upon the particular restorative sleep needs of the mammal. On the other hand, time of administration of the composition comprising the flavanol component may not be as important, and therefore this composition may be, for example, administered at any time that is convenient to the mammal. Of course, the invention fully contemplates concurrent administration of both of these compositions, which may be particularly convenient for the mammal, but the kit allows ease of flexibility and therefore greater opportunity for enhanced compliance with a treatment regimen.

The kits herein may be packaged in any manner, such as any manner that is ultimately convenient for the mammal. For example, the kit may offer a plurality of blister packs wherein each blister pack contains a daily dose of the composition comprising the melatonin component and the composition comprising the flavanol component. In this example, the compositions may be formulated as capsules or tablets, and each blister pack may contain one or more of each type of composition, depending upon the frequency of daily dose. Weekly, monthly, or other types of kits offering multiple doses of the discrete compositions may be provided.

### Methods of Use

The methods of the present invention comprise orally administering (i.e., through ingestion) a composition of the present invention to a mammal, preferably a human, to provide various health benefits, including inducing restorative sleep function, enhanced energy, and combinations thereof. The compositions of the present invention are most preferably ingested by consumers primarily desiring restorative sleep function and further desiring to complement this benefit with enhanced energy while taking advantage of the restorative actions of the mammalian body during rest sleep. The compositions of this invention may also be ingested as a supplement to normal dietetic requirements. Frequency of administration is not limited, however, such administration is typically at least once weekly, more preferably at least 3 times weekly, and most preferably at least once daily. Extent of need of restorative sleep function may dictate administration of at least a composition comprising the melatonin component.

As used herein, “restorative sleep function” refers to alleviation of any circadian rhythm phase-shifting effect, jet lag, winter depression, shift work-related desynchronies, sleep phase disorders, and other sleep disorders, improvement in sleep quality, improvement of sleep duration, and combinations thereof.

As used herein, the term “orally administering” with respect to the mammal (preferably, human) means that the mammal ingests or is directed to ingest (preferably, for the purpose of providing one or more of the health benefits described herein) one or more compositions of the present invention. In one embodiment, the composition is formulated as a tablet, capsule, food or beverage composition. Wherein the mammal is directed to ingest one or more of the compositions, such direction may be that which instructs and/or informs the user that use of the composition may and/or will provide one or more general health and/or general physiological benefits including, but not limited to, restorative sleep function, enhanced energy, and combinations thereof. For example, such direction may be oral direction (e.g., through oral instruction from, for example, a physician, health professional, sales professional or organization, and/or radio or television media (i.e., advertisement) or written direction (e.g., through written direction from, for example, a physician or other health professional (e.g., scripts), sales professional or organization (e.g., through, for example, marketing brochures, pamphlets, or other instructive paraphernalia), written media (e.g., internet, electronic mail, or other computer-related media), and/or packaging associated with the composition (e.g., a label present on a package containing the composition). As used herein, “written” means through words, pictures, symbols, and/or other visible descriptors. Such direction need not utilize the actual words used herein, for example, “sleep”, “restorative” “enhanced”, “energy”, “human”, or “mammal”, but rather use of words, pictures, symbols, and the like conveying the same or similar meaning are contemplated within the scope of this invention.

Since melatonin appears to be absorbed across almost all tissues, many routes of administration are possible. These include but are not limited to submucosal, sublingual, intranasal, ocular cul-de-sac, rectal, transdermal, buccal, intravenous, intramuscular, and subcutaneous routes of administration. A variety of administration means, including but not limited to capsules, tablets, suppositories, or any reservoir capable of containing and dispensing melatonin, are useful. In a preferred embodiment herein, the melatonin is administered orally.

The compositions herein may be, for example, formulated as an immediate or sustained release formulation. In one embodiment, a composition containing melatonin is in immediate release form. In another embodiment, a composition containing melatonin is in sustained release form (such as wherein the melatonin is continuously released over a set period of time). In yet another embodiment, a composition containing melatonin is in delayed release form (such as wherein the melatonin released at some time after administration). Wherein the invention is presented as a kit, the composition containing the flavanol component may be, for example, formulated as an immediate release composition, even wherein the composition containing the melatonin is formulated as a sustained release or delayed release formulation.

Further, the methods of the invention relate to the timing of the administration of the dosage of melatonin to the mammal. The timing of the melatonin in the mammal as described results in a specific phase shift (phase advance or phase delay) in the mammal's circadian rhythms.

### EXAMPLES

The following are non-limiting examples of the present compositions which are prepared utilizing conventional methods. The following examples are provided to illustrate the invention and are not intended to limit the scope thereof in any manner.

### Example 1

A kit is prepared, comprising a blister pack intended for seven day use. The blister pack contains seven compositions comprising a melatonin component and seven compositions comprising a flavanol component. All compositions are in tablet form.

The compositions comprising the melatonin component are in sustained release form and contain the following ingredients, at the indicated amounts:

| Component | (Wt %, approximate) |
|---|---|
| Melatonin | 0.5 |
| Lactose | 59 |
| Hydroxymethylcellulose | 30 |
| Microcrystalline Cellulose | 10 |
| Magnesium Stearate | 0.5 |

The compositions comprising the flavanol component contain:

| Component | Weight |
|---|---|
| Green Tea, powdered | 1000 mg |

Each day over a seven day period, a human in need of restorative sleep function and enhanced energy for daily activities ingests one tablet of the composition comprising the melatonin component and one tablet of the composition comprising the flavanol component. The human ingests the composition comprising the melatonin component about 4 hours prior to 11:00 PM, which is this particular human's desired sleep time. The human ingests the composition comprising the flavanol component during any convenient time of day, which is flexible throughout the dosing regimen. The human obtains additional kits and administers the compositions daily over an indefinite period of time.

### Example 2

A kit is prepared as in Example 1, except the composition comprising the melatonin component is a delayed release formulation. The composition comprising the melatonin component is in the form of a tablet, which is enterically coated with a methacrylic acid copolymer and simethicone mixture. The coating has the following approximate formula:

| Component | (Wt %, approximate) |
|---|---|
| Methacrylic Acid Copolymer | 99.85 |
| Simethicone | 0.15 |

Each day over a seven day period, a human in need of restorative sleep function and enhanced energy for daily activities ingests one tablet of the composition comprising the melatonin component and one tablet of the composition comprising the flavanol component. The human ingests the composition comprising the melatonin component about 8 hours prior to 11:00 PM, which is this particular human's desired sleep time. The human ingests the composition comprising the flavanol component during any convenient time of day, which is flexible throughout the dosing regimen. The human obtains additional kits and administers the compositions daily over an indefinite period of time.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.”

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A composition comprising:
(a) a melatonin component; and
(b) a flavanol component.

2. The composition according to claim 1 wherein the melatonin component is melatonin.

3. The composition according to claim 2 wherein the flavanol component comprises a compound selected from the group consisting of catechin, epicatechin, gallocatechin, epigallocatechin, epicatechin gallate, and epigallocatechin gallate and mixtures thereof.

4. The composition according to claim 2 wherein the flavanol component comprises green tea.

5. The composition according to claim 4 comprising from about 0.01 mg to about 100 mg of melatonin.

6. The composition according to claim 5 which is selected from the group consisting of tablets and capsules.

7. A kit comprising:
(a) a composition comprising a melatonin component; and
(b) a composition comprising a flavanol component.

8. The kit according to claim 7 wherein the melatonin component is melatonin.

9. The kit according to claim 8 wherein the flavanol component comprises a compound selected from the group consisting of catechin, epicatechin, gallocatechin, epigallocatechin, epicatechin gallate, epigallocatechin gallate, and mixtures thereof.

10. The kit according to claim 9 wherein the flavanol component comprises green tea.

11. The kit according to claim 10 wherein the composition comprising the melatonin component comprises from about 0.01 mg to about 100 mg of melatonin.

12. The kit according to claim 11 which each composition is, independently, selected from the group consisting of tablets and capsules.

13. A method selected from inducing restorative sleep function, and combinations thereof comprising administering to a mammal in need thereof the composition according to claim 1.

14. The method according to claim 13 wherein the administration is oral.

15. The method according to claim 14 wherein the composition is administered at least about 4 hours prior to desired sleep time of the mammal.

16. The method according to claim 15 wherein the composition is in sustained release form.

17. The method according to claim 16 wherein the composition is administered at least about 8 hours prior to desired sleep time of the mammal.

18. A method selected from inducing restorative sleep function, and combinations thereof comprising administering to a mammal in need thereof the composition comprising the melatonin component and the composition comprising the flavanol component.

19. The method according to claim 18 wherein the administration is oral.

20. The method according to claim 19 wherein the composition comprising the melatonin is administered at least about 4 hours prior to desired sleep time of the mammal.

21. The method according to claim 20 wherein the composition comprising the melatonin component is in sustained release form.

22. The method according to claim 21 wherein the composition comprising the melatonin component is administered at least about 8 hours prior to desired sleep time of the mammal.
